(19) United States
(12) Patent Application Publication
(10) Pub. No.: US 2026/0286290 A1
(43) Pub. Date: Sep. 24, 2026

(54) IN VITRO AGING MODEL HAVING MULTILAYER STRUCTURE AND MANUFACTURING METHOD THEREFOR

(71) Applicant: Korea Advanced Institute of Science and Technology, Daejeon (KR)

(72) Inventors: Pilnam KIM, Daejeon (KR); Insung YONG, Daejeon (KR)

(21) Appl. No.: 19/475,592

(22) PCT Filed: May 30, 2024
(86) PCT No.: PCT/KR2024/095850

§ 371 Date: Oct. 15, 2025

(30) Foreign Application Priority Data
May 30, 2023 (KR) 10-2023-0068882

(51) Int. Cl. C12N 5/00; G01N 33/50

(52) U.S. Cl. CPC C12N 5/0068; G01N 33/5082; C12N 2533/52; C12N 2533/54; C12N 2533/90

(57) ABSTRACT

The present invention relates to an in vitro aging model having a multilayer structure and, more specifically, to an in vitro aging model for use in tissue aging research, including an extracellular matrix with a multilayer structure and to a manufacturing method thereof. The majority of the cellular structures of the individual are organized in three dimensions, which results in complex intercellular interactions that cannot be mimicked in two-dimensional monolayer cell cultures. Therefore, in order to develop highly bio-responsive technology in various fields such as research on diseases, customized medicine, and new drug development, there is an increasing need for an in vitro model that similarly mimics the shape of a three-dimensional cell structure. Since the in vitro aging model of the present invention implements the multi-layer ECM structure as it is, the in vitro aging model can mimic the three-dimensional structure of various tissues according to the type of cells to be applied. In particular, since being similar to the ECM structure of skin tissues, the in vitro aging model is expected to be largely used in skin aging research.

### TECHNICAL FIELD

The present invention relates to an in-vitro aging model having a multilayer structure, and more specifically, to an in-vitro model including an extracellular matrix of a multilayer structure, which is used for tissue aging research, and to a method for producing the same.

### BACKGROUND ART

Most cellular structures of animal and plant organisms, including humans, are organized in three dimensions, which results in complex intercellular interactions that cannot be mimicked in two-dimensional monolayer cell cultures. Therefore, in various fields such as disease research, personalized medicine, and new drug development, the need for in-vitro models that closely mimic the morphology of three-dimensional cellular structures has been increasing for the development of technologies with high biological relevance.

In particular, the skin is an organ in which the maintenance and restoration of the three-dimensional tissue structure are very important. In modern times, as industrial society has developed and social activities have increased, self-awareness to actively express oneself has risen, leading to increased interest in beauty and suppression of skin aging. Collapse of the three-dimensional structure in skin tissue causes wrinkles, decreased elasticity, and dryness, so it is not an exaggeration to say that the essence of skin aging is determined by the connective tissue in the dermis, namely the extracellular matrix (ECM).

Accordingly, the present invention was devised to solve the above problems and relates to an in-vitro aging model including an extracellular matrix with a multilayer structure, and a method for producing the same. Since the in-vitro aging model of the present invention faithfully implements the multilayer ECM structural form, it can mimic three-dimensional structures of various tissues depending on the type of cells applied, and in particular, it is expected to be greatly utilized in skin aging research because its ECM form is similar to that of skin tissue.

### DISCLOSURE

### Technical Problem

An object of the present invention is to provide a multilayer extracellular matrix structure, namely, an in-vitro aging model having a multilayer structure.

Another object of the present invention is to provide a method for producing the in-vitro aging model having a multilayer structure.

A further object of the present invention is to provide a method for screening candidate substances for inhibiting tissue aging using the in-vitro aging model having a multilayer structure.

Another object of the present invention is to provide a method for evaluating the effect of substances for inhibiting tissue aging using the in-vitro aging model having a multilayer structure.

Another object of the present invention is to provide a method for researching tissue aging using the in-vitro aging model having a multilayer structure.

The tissue is preferably skin tissue but is not limited thereto.

However, objects to be achieved by the present disclosure are not limited to the objects mentioned above, and other objects not mentioned above may be clearly understood by those skilled in the art from the following description.

### Technical Solution

Hereinafter, various embodiments described herein will be described with reference to figures. In the following description, numerous specific details are set forth, such as specific configurations, compositions, and processes, etc., in order to provide a thorough understanding of the present disclosure. However, certain embodiments may be practiced without one or more of these specific details, or in combination with other known methods and configurations. In other instances, known processes and preparation techniques have not been described in particular detail in order not to unnecessarily obscure the present disclosure. Reference throughout this specification to “one embodiment” or “an embodiment” means that a particular feature, configuration, composition, or characteristic described in connection with the embodiment is included in at least one embodiment of the present disclosure. Thus, the appearances of the phrase “in one embodiment” or “an embodiment” in various places throughout this specification are not necessarily referring to the same embodiment of the present disclosure. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present disclosure pertains.

Throughout the specification, when a part is said to “comprise” a certain component, this means that other components may be further included unless otherwise specifically stated, rather than excluding other components.

Histologically, the skin is composed of three layers: the epidermis, which is epithelial tissue, the dermis, which is connective tissue located below the epidermis, and the subcutaneous fat layer. In particular, the dermis occupies most of the skin tissue and plays an important role in maintaining the actual structure and function. As aging progresses, the cells and various components constituting the dermis change quantitatively and qualitatively, resulting in aging phenomena. When aging occurs in the dermis, visible changes such as decreased skin elasticity, formation of deep wrinkles, and increased skin dryness occur. Therefore, it is no exaggeration to say that the essence of skin aging is determined by the connective tissue in the dermis, namely the extracellular matrix (ECM). The ECM, which constitutes most of the dermis, is a complex mesh-like ground substance that fills the space between cells outside the cells, and as a part of the tissue that is not a cell, it provides an external environment for the cells to survive healthily. The components constituting the dermal ECM are synthesized inside fibroblasts and secreted through extracellular release. Representative examples include fibrous proteins such as collagen and elastin, amorphous matrix components such as proteoglycan complexes, and glycoproteins such as fibronectin and laminin that connect cells to the matrix. A summary of the ECM components and characteristics is as follows.

### [1] Amorphous Matrix

### \textcircled{1} Glycosaminoglycan (GAG)

Six types: hyaluronic acid (HA), chondroitin sulfate (CS), dermatan sulfate (DS), heparan sulfate (HS), heparin (HP), and keratan sulfate (KS).
Negatively charged due to carboxyl and sulfate groups and performs a water retention function by easily binding to water molecules.
GAGs generally cannot exist alone and are present in combination with other proteins.

### \textcircled{2} Proteoglycan (PG)

A gel-type complex of GAG and a specific core protein.
Acts as a cushion filling the spaces between fibrous proteins.
Retains water and facilitates free movement of various nutrients.
Involved in signal transmission between cells or between various fibers.

### [2] Fibrous Structural Proteins

### \textcircled{1} Collagen

A triple-helical structure formed by linking various amino acids in a fibrous shape.
Constitutes most of the dermis, with type I and type III collagen being representative.
Provides tension supporting the firm structure of the skin.

### \textcircled{2} Elastin

Functions like a spring, providing elasticity and extensibility necessary for the skin to recover to its original shape.

### [3] Fibrous Adhesive Proteins

Fibronectin
A glycoprotein that allows cells and matrix to be interconnected.
Directly binds to integrin receptors on the cell membrane surface to induce activation.

### [4] Others

Growth factors (EGF, FGF, VEGF, etc.), integrin receptors, matrix metalloproteinases (MMP), etc.

In summary, the ECM in the dermis acts as a physical support structure filling the spaces between cells, is involved in supplying nutrients and water, and regenerating damaged tissue, and controls interaction and communication with cells. Therefore, collapse of the ECM structure is directly connected to skin aging.

Accordingly, the in-vitro aging model including a multilayer ECM structure provided by the present invention is expected to be useful for skin aging research.

According to one embodiment of the present invention, a method for manufacturing a multilayer extracellular matrix structure, comprising: (b) a step of culturing cells on the substrate of step (a); (c) a step of removing components other than extracellular matrix from the substrate of step (b); and (d) a step of glycation of step (c).

The multilayer extracellular matrix structure produced by the above method is considered to have the same meaning as the “in-vitro aging model having a multilayer structure” described in the present invention.

The hydrophobic substrate is preferably a glass substrate, but is not limited thereto. The hydrophobic substrate may be converted to a hydrophilic surface by a step of performing oxygen plasma treatment. The oxygen plasma treatment may be performed at an intensity of 30 W for 1 minute, but is not limited thereto. When a substrate having a hydrophilic surface is used, the step of performing oxygen plasma treatment may be omitted.

The method for manufacturing the multilayer extracellular matrix structure of the present invention may further comprise, prior to coating the hydrophobic substrate with a cell adhesion molecule, a step of treating the substrate with oxygen plasma and coating it with an acrylate-based polymer resin. In this case, the acrylate-based polymer resin was adopted because it satisfies the effects of firmly fixing the lowermost extracellular matrix to the glass substrate (adhesion strength), exhibiting excellent biocompatibility so as not to interfere with cell viability (biocompatibility), and providing an appropriate balance between flexibility and rigidity (mechanical properties) to support the multilayer extracellular matrix structure. The acrylate-based polymer resin may be one or more selected from the group consisting of methyl acrylate, ethyl acrylate, butyl acrylate, 2-carboxyethyl acrylate, methyl methacrylate, ethyl methacrylate, butyl methacrylate, hydroxyalkyl acrylate, hydroxyethyl methacrylate, hydroxyalkyl methacrylate, acrylamide, aminoalkyl acrylate, aminoalkyl methacrylate, tetra(ethylene glycol) dimethacrylate, hydroxypropyl methacrylate, 2-hydroxyethyl acrylate, polyurethane acrylate, poly(methyl methacrylate), poly(glycidyl methacrylate), 1-vinyl-2-pyrrolidone, di(ethylene glycol)ethyl ether methacrylate, ethylene glycol methyl ether methacrylate, 3-sulfopropyldimethyl-3-methacrylamidopropyl-ammonium, acrylic acid, and methacrylic acid, but is not limited thereto.

The cell adhesion molecule may be one or more selected from the group consisting of collagen, fibronectin, laminin, elastin, gelatin, poly-L-lysine, Matrigel, proteoglycan, and glycosaminoglycan, and is preferably fibronectin.

Cells cultured on the substrate coated with the cell adhesion molecule are not limited thereto, but are preferably stromal cells that constitute fibrous connective tissue. In this case, the stromal cells may be one or more selected from the group consisting of fibroblast, chondroblast, osteoblast, neuroglial cell, adipocyte, macrophage, and plasma cell.

The method for producing the multilayer extracellular matrix structure of the present invention is characterized in that the steps of (d) culturing cells on the substrate of step (c) and (e) removing components other than the extracellular matrix from step (d) are repeatedly performed to form a multilayer extracellular matrix. When steps (d) and (e) are performed n times, the multilayer extracellular matrix structure is understood to form n layers. Preferably, the multilayer extracellular matrix structure of the present invention may be formed by repeating steps (d) and (e) three or more times to form three or more layers. More preferably, steps (d) and (e) may be repeated four or more times, and most preferably, steps (d) and (e) may be repeated five or more times.

The method for producing the multilayer extracellular matrix structure of the present invention has an important feature of including a glycation process. In this case, the glycation may be performed using ribose or glucose, and is preferably performed using ribose, and may be performed for at least 1 day (24 hours), at least 3 days (72 hours), at least 1 week, at least 2 weeks, or at least 3 weeks, preferably for at least 3 days (72 hours), more preferably for at least 1 week, and most preferably for at least 3 weeks, but is not limited thereto.

The multilayer extracellular matrix structure produced by the method of the present invention is characterized in that it does not contain nucleic acids and/or lipids by removing components other than the extracellular matrix. Here, the term “components other than the extracellular matrix” means all components of the cell, including nucleic acids, proteins, lipids, carbohydrates, and solution components, except for those required for the synthesis and maintenance of the extracellular matrix.

In another embodiment of the present invention, the present invention provides a multilayer extracellular matrix structure including multilayer extracellular matrix formed on a substrate.

The multilayer extracellular matrix structure of the present invention may be produced by the “method for producing a multilayer extracellular matrix structure” of the present invention described above, and may be understood to have the same meaning as the in-vitro aging model having a multilayer structure in the present invention.

The substrate is preferably a glass substrate, but is not limited thereto. When the surface of the substrate is hydrophobic, the substrate may be subjected to oxygen plasma treatment to convert the surface into a hydrophilic surface.

The multilayer extracellular matrix structure may additionally include an acrylate-based polymer resin in order to form multilayer extracellular matrix on the substrate. In this case, the acrylate-based polymer resin may be one or more selected from the group consisting of methyl acrylate, ethyl acrylate, butyl acrylate, 2-carboxyethyl acrylate, methyl methacrylate, ethyl methacrylate, butyl methacrylate, hydroxyalkyl acrylate, hydroxyethyl methacrylate, hydroxyalkyl methacrylate, acrylamide, aminoalkyl acrylate, aminoalkyl methacrylate, tetra(ethylene glycol) dimethacrylate, hydroxypropyl methacrylate, 2-hydroxyethyl acrylate, polyurethane acrylate, poly(methyl methacrylate), poly(glycidyl methacrylate), 1-vinyl-2-pyrrolidone, di(ethylene glycol)ethyl ether methacrylate, ethylene glycol methyl ether methacrylate, 3-sulfopropyldimethyl-3-methacrylamidopropyl-ammonium, acrylic acid, and methacrylic acid, but is not limited thereto.

The multilayer extracellular matrix structure may additionally include a cell adhesion molecule in order to form multilayer extracellular matrix on the substrate. In this case, the cell adhesion molecule may be one or more selected from the group consisting of collagen, fibronectin, laminin, elastin, gelatin, poly-L-lysine, Matrigel, proteoglycan, and glycosaminoglycan, and is preferably fibronectin.

The multilayer extracellular matrix included in the multilayer extracellular matrix structure is not limited thereto, but may be produced from stromal cells that constitute fibrous connective tissue. In this case, the stromal cells may be one or more selected from the group consisting of fibroblast, chondroblast, osteoblast, neuroglial cell, adipocyte, macrophage, and plasma cell.

The multilayer extracellular matrix structure of the present invention preferably comprises three or more layers, more preferably four or more layers, and most preferably five or more layers, and may additionally be glycated.

The multilayer extracellular matrix structure of the present invention is characterized in that it does not contain nucleic acids and/or lipids by removing components other than the extracellular matrix. Here, the term “components other than the extracellular matrix” means all components of the cell, including nucleic acids, proteins, lipids, carbohydrates, and solution components, except for those required for the synthesis and maintenance of the extracellular matrix.

The multilayer extracellular matrix structure of the present invention may additionally be glycated, and in this case, the glycation may be performed using ribose or glucose for at least 1 day (24 hours), at least 3 days (72 hours), at least 1 week, at least 2 weeks, or at least 3 weeks.

The multilayer extracellular matrix structure of the present invention can be used for research on tissue aging. In this case, the structure may be used as an in-vitro model for research on aging of various tissues depending on the type of cells applied. For example, when skin cells are cultured on the multilayer extracellular matrix structure of the present invention, it becomes an in-vitro model for research on skin tissue aging; when adipocytes are cultured, it becomes an in-vitro model for research on adipose tissue aging; when muscle cells are cultured, it becomes an in-vitro model for research on muscle tissue aging; and when hepatocytes are cultured, it becomes an in-vitro model for research on liver tissue aging. As a preferred embodiment, the multilayer extracellular matrix structure of the present invention is used as an in-vitro model for skin aging research, but is not limited thereto.

In another embodiment of the present invention, the present invention provides a method for screening candidate substances for inhibiting tissue aging, comprising: (a) a step of producing a multilayer extracellular matrix structure including multilayer extracellular matrix formed on a substrate; (b) a step of treating the structure with a candidate substance for inhibiting tissue aging; and (c) a step of confirming changes in the structure.

As used herein, “screening” means selecting, by a specific operation or evaluation method, a substance having a particular desired property from a group of candidates consisting of various substances.

The multilayer extracellular matrix structure used in the screening method of the present invention may be produced by the “method for producing a multilayer extracellular matrix structure” of the present invention described above, and may be understood to have the same meaning as the in-vitro aging model having a multilayer structure in the present invention.

Restrictions on each part of the “multilayer extracellular matrix structure” and the “method for producing a multilayer extracellular matrix structure” are as described above in the specification of the present invention and are omitted hereinafter to avoid excessive complexity of the specification.

In the screening method, if, after treating the candidate substance for inhibiting tissue aging, stiffness and fiber thickness in the sample are significantly decreased, or adhesion force and pore size are significantly increased compared with before treatment, the candidate substance for inhibiting tissue aging may be determined to be effective for inhibiting tissue aging.

Conversely, if, after treating the multilayer extracellular matrix structure of the present invention with a candidate substance for promoting tissue aging, stiffness and fiber thickness in the sample are significantly increased, or adhesion force and pore size are significantly decreased compared with before treatment, the candidate substance for promoting tissue aging may be determined to be effective for promoting tissue aging.

By appropriately adjusting and testing the candidate substance for inhibiting tissue aging and the candidate substance for promoting tissue aging, the multilayer extracellular matrix structure of the present invention can be used for research on tissue aging.

In addition, by confirming the changes in the multilayer extracellular matrix structure in this manner, the development of a pharmaceutical composition, a food composition, or a cosmetic composition for inhibiting or improving tissue aging becomes possible. Although not limited thereto, the active ingredient of the pharmaceutical composition, food composition, or cosmetic composition may be a candidate substance for inhibiting tissue aging derived through the screening method of the present invention.

In this case, the pharmaceutical composition may, for example, be for inhibiting or improving skin aging and may further be for preventing or treating various skin diseases caused by skin aging. The pharmaceutical composition may include, as an active ingredient, the candidate substance for inhibiting skin aging, and may additionally include proteins involved therein and a pharmaceutically acceptable carrier, excipient, or diluent. The term “pharmaceutically acceptable” carrier or excipient means one that is approved by a governmental regulatory agency, or is listed in a pharmacopeia generally approved for use in vertebrates, and more specifically, in humans. Considering that the pharmaceutical composition of the present invention is applied to skin tissue, for parenteral administration the pharmaceutical composition may be in the form of a suspension, solution, or emulsion in an oily or aqueous carrier and may be manufactured in solid or semi-solid form. In addition, the pharmaceutical composition of the present invention may include formulation agents such as suspending agents, stabilizers, solubilizers, and/or dispersants, and may be sterilized. The pharmaceutical composition may be stable under conditions of manufacture and storage and may be preserved against the contaminating action of microorganisms such as bacteria or fungi. Alternatively, the pharmaceutical composition of the present invention may be in a sterile powder form for reconstitution with an appropriate carrier before use. The pharmaceutical composition may be present in a unit-dose form, in a microneedle patch, in an ampule, or in another unit-dose container, or in a multi-dose container. Alternatively, the pharmaceutical composition may be stored in a freeze-dried (lyophilized) state requiring addition of sterile water for injection immediately before use. Immediate-use injectable solutions and suspensions may be prepared in the form of sterile powders, granules, or tablets. In some non-limiting embodiments, the pharmaceutical composition of the present invention may be formulated and/or included in a liquid in the form of fine particles. In some non-limiting embodiments, the pharmaceutical composition of the present invention may contain the pharmaceutically acceptable compound(s) and/or mixture(s) thereof at a concentration ranging from 0.001 to 100,000 U/kg. In addition, in some non-limiting embodiments, the pharmaceutical composition of the present invention may include an appropriate excipient such as a preservative, suspending agent, additional stabilizer, dye, buffer, antibacterial agent, antifungal agent, and isotonic agent, for example, sugar or sodium chloride. As used herein, the term “stabilizer” refers to a compound that is optionally used in the pharmaceutical composition of the present invention to increase shelf life. In a non-limiting embodiment, the stabilizer may be a sugar, an amino acid, or a polymer.

In addition, in the present invention, the food composition may, for example, be for inhibiting or improving skin aging, and specifically, may be for skin antioxidation, whitening, anti-inflammation, and wrinkle improvement. As used herein, “antioxidation” means suppression of oxidation, that is, the concept of suppressing cellular aging. Oxygen introduced into the body through respiration not only produces energy necessary for the human body but also generates free radicals, which are harmful excess oxygen species. Free radicals attack normal cells in the body and act as a cause of aging and various diseases. Therefore, removing free radicals is a way to prevent cellular oxidation (aging), and suppressing such cellular oxidation is antioxidation. As used herein, “whitening” refers to a function of preventing excessive deposition of melanin pigment on the skin by suppressing an increase of melanocytes caused by continuous exposure of the skin to ultraviolet rays, or of lightening melanin pigment already deposited. Accordingly, it is possible to suppress the formation of blemishes, freckles, lentigines, and melanomas caused by excessive deposition of melanin pigment. As used herein, “anti-inflammation” means an effect of calming and resisting inflammation. Anti-inflammation in the skin can be regarded as protecting and stabilizing the skin, and performing disinfection and sterilization. As used herein, “wrinkle” is meant to include externally visible outer wrinkles and inner wrinkles formed at the dermal-epidermal junction. Further, “anti-wrinkle” or “wrinkle suppression” means delaying, suppressing, or improving the formation of wrinkles. The food composition of the present invention including a candidate substance for inhibiting skin aging derived through the screening method of the present invention may be manufactured in the form of various food types, for example, beverages, chewing gum, tea, vitamin complexes, powders, granules, tablets, capsules, confectionery, rice cakes, or bread. Since it is desirable that the food ingredients are composed of natural foods, it can be used safely with almost no toxicity or side effects even when taken for long-term preventive purposes. When the composition of the present invention is included in a food composition, the amount may be added at a ratio of 0.1 to 100% by total weight. When the food composition is manufactured in the form of a beverage, there are no particular limitations other than containing the food composition at the indicated ratio, and it may additionally include various flavoring agents or natural carbohydrates like those contained in ordinary beverages. That is, as natural carbohydrates, it may include monosaccharides such as glucose, disaccharides such as fructose, sucrose, and polysaccharides such as dextrin or cyclodextrin, as well as sugar alcohols such as xylitol, sorbitol, and erythritol. Examples of the flavoring agents include natural flavoring agents (thaumatin, stevia extract such as rebaudioside A, glycyrrhizin, etc.) and synthetic flavoring agents (saccharin, aspartame, etc.). In addition, the food composition of the present invention may include various nutrients, vitamins, minerals (electrolytes), synthetic and natural flavoring agents, coloring agents, pectic acid and salts thereof, alginic acid and salts thereof, organic acids, protective colloidal thickeners, pH regulators, stabilizers, preservatives, glycerin, alcohol, and carbonating agents used in carbonated beverages. These components may be used independently or in combination. Although the ratio of these additives is not particularly critical, it is generally selected in the range of 0.1 to 100 parts by weight per 100 parts by weight of the composition of the present invention.

In addition, in the present invention, the cosmetic composition may, for example, be for inhibiting or improving skin aging, and specifically, may be for skin antioxidation, whitening, anti-inflammation, and wrinkle improvement. The cosmetic composition of the present invention including a candidate substance for inhibiting skin aging derived through the screening method of the present invention may be manufactured in the form of skin lotion, nourishing lotion, nourishing essence, massage cream, beauty bath additive, body lotion, body milk, bath oil, baby oil, baby powder, shower gel, shower cream, sunscreen lotion, sunscreen cream, suntan cream, skin lotion, skin cream, cosmetics for ultraviolet protection, cleansing milk, depilatory {for cosmetic use}, face and body lotion, face and body cream, skin whitening cream, hand lotion, hair lotion, cosmetic cream, jasmine oil, bath soap, liquid soap, beauty soap, shampoo, hand cleanser, medicated soap {non-medical}, cream soap, facial wash, hair rinse, cosmetic soap, tooth whitening gel, or toothpaste. For this purpose, the composition of the present invention may further include an appropriate carrier, excipient, or diluent conventionally used for the manufacture of a cosmetic composition. The carriers, excipients, or diluents that may further be added to the cosmetic composition of the present invention may be one or more selected from the group consisting of purified water, oils, waxes, fatty acids, fatty alcohols, fatty acid esters, surfactants, humectants, thickeners, antioxidants, viscosity stabilizers, chelating agents, buffers, and lower alcohols, but are not limited thereto. In addition, as needed, the cosmetic composition may include whitening agents, moisturizers, vitamins, ultraviolet screening agents, perfumes, dyes, antibiotics, antibacterials, and antifungal agents. Examples of the oils include hydrogenated vegetable oil, castor oil, cottonseed oil, olive oil, coconut oil, jojoba oil, and avocado oil. Examples of the waxes include beeswax, paraffin wax, carnauba, candelilla, montan, ceresin, liquid paraffin, and lanolin. Examples of the fatty acids include stearic acid, linoleic acid, linolenic acid, and oleic acid. Examples of the fatty alcohols include cetyl alcohol, octyldodecanol, oleyl alcohol, panthenol, lanolin alcohol, stearyl alcohol, and hexadecanol. Examples of the fatty acid esters include isopropyl myristate, isopropyl palmitate, and butyl stearate. The surfactants may be cationic surfactants, anionic surfactants, and nonionic surfactants known in the art, and preferably are surfactants derived from natural products whenever possible. In addition, humectants, thickeners, antioxidants, and the like widely known in the field of cosmetics may be included, and the kinds and amounts thereof follow those known to a person skilled in the art.

Hereinafter, the present invention will be described in detail with reference to examples.

### Advantageous Effects

Since the in-vitro aging model of the present invention faithfully implements the multilayer ECM structural form, it can mimic the three-dimensional structures of various tissues depending on the type of cells applied, and in particular, because its ECM form is similar to that of skin tissue, it is expected to be greatly utilized for skin aging research.

### DESCRIPTION OF DRAWINGS

FIGS. 1a to 1e are schematic diagrams illustrating the manufacturing steps of the in-vitro aging model having a multilayer structure (multilayer ECM structure) produced according to the present invention.

FIGS. 2a and 2b are views showing changes when the cell culture and cellular component (CC) removal process in the multilayer ECM structure of the present invention are performed once, twice, three times, four times, or five times.

FIGS. 3a and 3b are views showing changes according to the performance of the cellular component (CC) removal process during the production of the multilayer ECM structure of the present invention.

FIG. 4 is a view subdividing and showing the composition ratios of ECM proteins and matrisome including ECM-related proteins in the multilayer ECM structure of the present invention.

FIGS. 5a to 5f are views showing relative quantitative comparisons of representative proteins of each group of matrisomes constituting the multilayer ECM structure of the present invention.

FIGS. 6a to 6d are views showing changes according to the performance of glycation during the production of the multilayer ECM structure of the present invention.

FIGS. 7 and 8 are views showing results of proteomic analysis after glycation treatment of the ECM structure of the present invention with ribose. Specifically, FIG. 7 shows a result of selecting the top 14 proteins according to the degree of glycation among matrisomes present in the multilayer ECM structure, and FIG. 8 shows a result of confirming, by proteomic mass spectrometry, amino acid residues at which advanced glycation end products are present and the types of advanced glycation end products at each amino acid residue for each protein constituting the multilayer ECM structure.

FIGS. 9 to 11 are views showing results of proteomic analysis of the ECM structure glycated with ribose according to the present invention. Specifically, FIG. 9 shows amino acid residues at which advanced glycation end products were identified in the full-length protein of fibronectin, FIG. 10 shows amino acid residues at which advanced glycation end products were identified in the full-length protein of collagen type I, and FIGS. 11a and 11b show results of GO (Gene Ontology) analysis using the top 14 glycated proteins described in FIG. 7.

FIGS. 12 to 15 are views showing results of proteomic analysis of the ECM structure glycated with glucose according to the present invention. Specifically, FIG. 12 shows a result of selecting the top 17 proteins according to the degree of glycation among matrisomes present in the multilayer ECM structure, and FIG. 13 shows a result of confirming advanced glycation end products by proteomic mass spectrometry. FIG. 14 shows amino acid residues at which advanced glycation end products were identified in the full-length protein of fibronectin, and FIG. 15 shows amino acid residues at which advanced glycation end products were identified in the full-length protein of collagen type I.

FIGS. 16 and 17 are views showing results of analyzing the effect of coating with an acrylate-based polymer resin in the production of the multilayer ECM structure of the present invention. Specifically, FIG. 16 shows a result of manufacturing a multilayer structure without coating any polymer, and FIG. 17 shows a result of manufacturing a multilayer structure by coating with PDMS, which is a non-acrylate-based polymer resin.

### BEST MODE

Glass coverslips were treated with oxygen plasma, then coated with an acrylate-based polymer resin, additionally coated with fibronectin, and NIH/3T3 cells were seeded thereon. The cells were cultured until they formed 100% confluency on the surface of the cover glass, and then the cellular components (CC) were removed to extract only highly purified extracellular matrix (ECM). This was washed at least three times with DPBS (Dulbecco's phosphate buffer saline), cells were recultured on the single-layer ECM, and when the cells reached 100% confluency, the ECM was re-extracted. By repeating the steps of cell culture and ECM extraction, an in-vitro aging model having a multilayer structure was produced. Finally, the produced multilayer ECM was reacted for 3 weeks at 37° C. in ribose dissolved in DPBS while maintaining the temperature condition, thereby producing a glycated multilayer in-vitro aging model.

### MODE FOR INVENTION

Hereinafter, the present disclosure will be described in detail with reference to the following examples. However, the following examples are merely illustrative of the present disclosure, and the content of the present disclosure is not limited by the following examples.

### EXAMPLE

### Example 1: Production of an In-Vitro Aging Model Having a Multilayer Structure

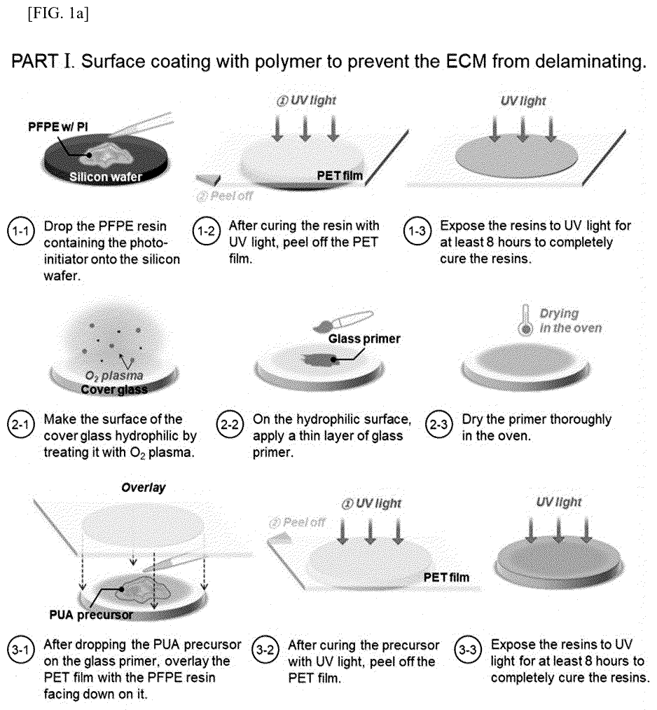

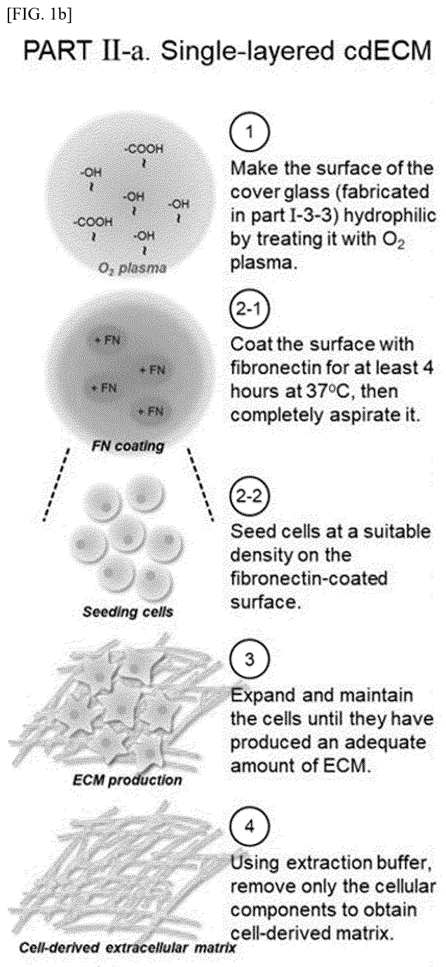

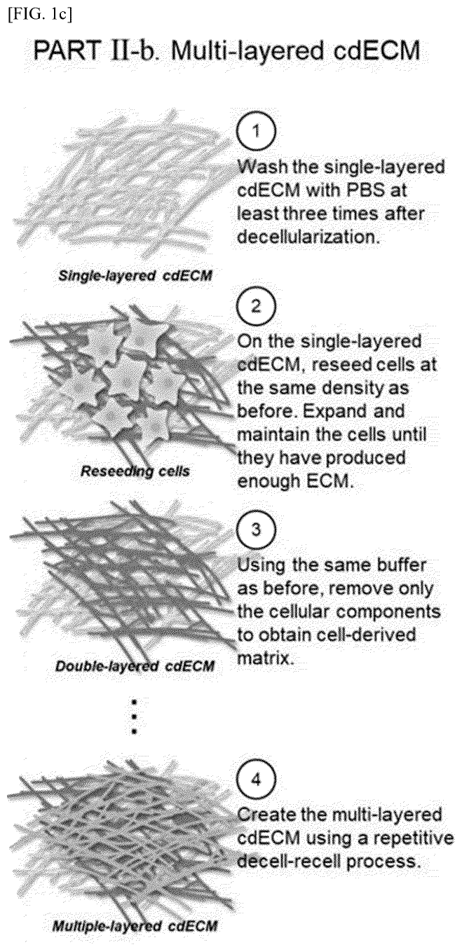

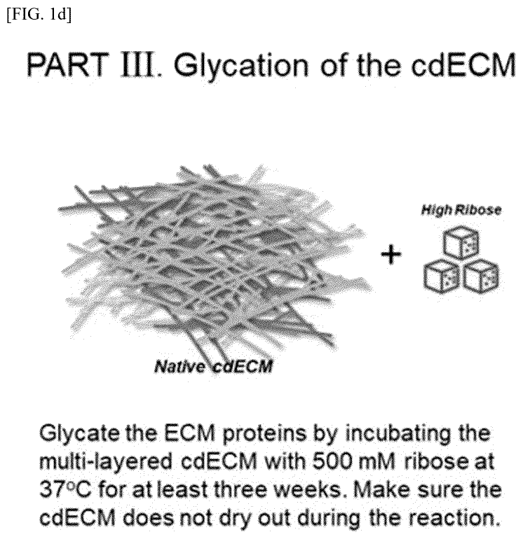

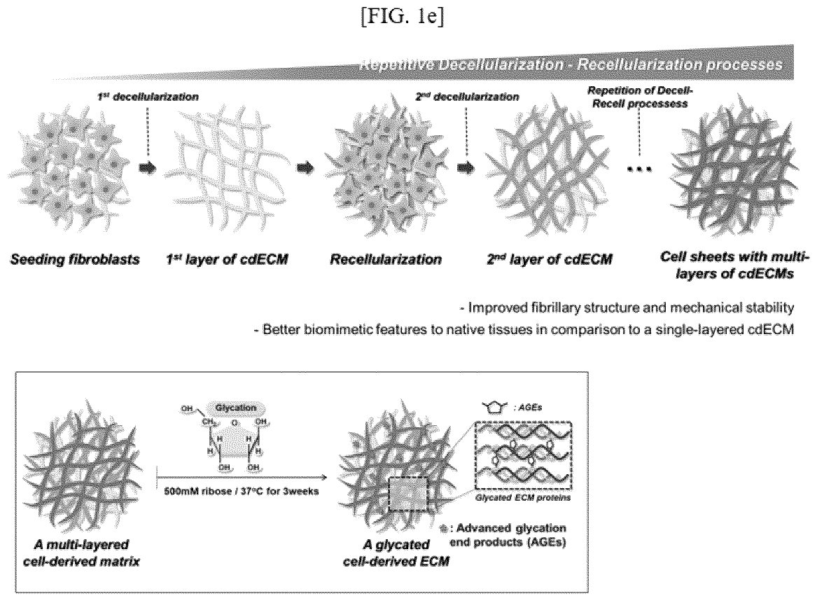

Glass coverslips were treated with oxygen plasma at an intensity of 30 W for 1 minute to temporarily convert the hydrophobic surface into a hydrophilic surface. An acrylate-based polymer resin (PUA) was coated thereon, then additionally coated with fibronectin for 4 hours or longer at 37° C., placed in a 35π culture dish (petri dish), and NIH/3T3 cells at 1×10⁴/cm² were seeded. The cells were cultured so as to form 100% confluency on the surface of the cover glass, and an extraction buffer containing 0.1% SDS (sodium dodecyl sulfate), 0.1% Triton X-100, and 0.001% PLL (poly-L-lysine) was then treated to remove cellular components (CC) and to extract only highly pure extracellular matrix (ECM). This was washed 3 or more times with DPBS (Dulbecco's phosphate buffer saline), cell culture was re-performed on the single-layer ECM, and when the cells formed 100% confluency, the ECM was re-extracted. By the method of repeating the above cell-culture and ECM-extraction steps, an in-vitro aging model having a multilayer structure was manufactured. Finally, the produced multilayer ECM was reacted for 3 weeks while maintaining a condition of 37° C. in 500 mM ribose, or glucose, dissolved in DPBS to produce a glycated model. The method for manufacturing the in-vitro aging model having a multilayer structure of the present invention was schematically shown in FIGS. 1a to 1e.

### Example 2: Verification of Usefulness of the Multilayer In-Vitro Aging Model

### Experimental Methods

### 1. Immunostaining

After washing the sample with DPBS, it was fixed at room temperature for 20 minutes with a 4% paraformaldehyde solution, and then permeabilized at room temperature for 15 minutes with a 0.15% (v/v) Triton X-100 solution to impart permeability to the sample. This was washed, and, to prevent nonspecific binding, blocking treatment was performed at room temperature for 1 hour with a 1% (w/v) bovine serum albumin (BSA) solution. The primary antibody was diluted in a 1% (w/v) BSA solution and treated at 4° C. for 16-24 hours, and the secondary antibody was treated at room temperature for 1 hour 30 minutes-2 hours. Thereafter, phalloidin was treated at a concentration of 5 μg/ml at room temperature for 45 minutes, and DAPI was treated at a concentration of 4 μg/ml at room temperature for 15 minutes. After washing and mounting, it was completely dried and stored at −20° C. or −80° C.

### 2. Scanning Electron Microscope Imaging

After washing the sample with DPBS, primary fixation was performed at room temperature for 30 minutes with a 2% (v/v) glutaraldehyde solution, and secondary fixation was performed at room temperature for 30 minutes with a 1% (v/v) osmium tetroxide solution. Thereafter, the sample was dehydrated by sequentially permeating the sample at room temperature for 10 minutes each with 25%, 50%, 75%, 95%, 100%, and 100% (v/v) ethanol solutions, and subjected to substitution by treating with a tert-butyl alcohol solution at room temperature for 30 minutes each, three times, followed by freeze-drying to completely dry the sample. This was firmly attached to a scanning electron microscope stub using carbon double-sided tape and silver paste, platinum-coated at 20 mA for 1 minute 30 seconds, and then scanned by a scanning electron microscope.

### 3. Proteomic Mass Analysis of Multilayer Cell-Derived Extracellular Matrix

Cell-derived extracellular matrix was harvested from the cover glass using a cell scraper, dissolved in a buffer consisting of 5% (w/v) SDS (sodium dodecyl sulfate), 50 mM TEAB (triethylammonium bicarbonate), and pH 8.5, and the supernatant centrifuged at 13,000 rpm for 8 minutes was quantified for protein by the BCA (bicinchoninic acid) method. The sample was reduced by treating 5 mM TCEP (Tris(2-carboxyethyl)phosphine) for 10 minutes, alkylated using iodoacetamide (IAA) diluted in isopropyl alcohol, and diluted with a phosphoric acid solution. Protein of 300 μg was mixed with a binding/wash buffer consisting of a TEAB solution diluted to 100 mM in 90% methanol, and the process of filtering with an S-Trap column was repeated three times to purify the protein; then trypsin diluted with a 50 mM TEAB solution was treated at 47° C. for 1-2 hours or at 37° C. for 16 hours to digest the protein. Thereafter, the sample was diluted with a 50 mM TEAB solution and subjected to a first centrifugation at 4,000 rcf, a 0.2% formic acid solution was added and a second centrifugation was performed, a 50% diluted acetonitrile solution was added and a third centrifugation was performed, the eluted peptides were dried, and then re-suspended in a 0.1% formic acid solution for mass analysis. The above sample was analyzed for peptides by reversed-phase high performance liquid chromatography (Reversed-phase-High Performance LC, RP-HPLC) using a 75 μm×50 cm analytical column, and MS/MS analysis data were integrated and analyzed through IP2 software based on the Uniprot database. Specifically, the data were recognized on a peak basis, information such as mass/charge (m/z) of the peaks was extracted, information such as amino acid sequences was checked from the database, and protein identification was performed by comparing with spectra of the obtained peptides. A precursor mass error of 4.5 ppm and a fragment ion mass error of 20 ppm were used, and in peptide spectrum matching and protein identification utilizing a target decoy strategy, the false discovery rate (FDR) was set to 0.01.

### 4. Measurement of Elastic Modulus of Multilayer Cell-Derived Extracellular Matrix

Pretreatment for elastic modulus measurement was performed in the same manner as up to the dehydration process of the scanning electron microscope imaging above. A cantilever having an appropriate spring constant was fixed to a metal chip carrier and mounted on an atomic force microscope to measure the force-distance curve of the sample. Thereafter, the completely dried sample by freeze-drying was placed on the sample stage of the atomic force microscope, the super-luminescent diode was aligned to the end of the cantilever and the center of the position-sensitive detector, and contact mode was executed. The probe was moved close to the sample, the distance between the sample and the probe was precisely adjusted at an appropriate position, setting values such as the z-axis travel range, speed, and force limit of the probe were input, and force-distance was repeatedly measured at multiple positions within the sample. The measured force-distance graph data were substituted into the Hertz model conversion formula to calculate the elastic modulus at each position within the sample. The Poisson's ratio was calculated as 0.5.

### Experimental Results

### 1. Confirmation of Changes in ECM Stacking

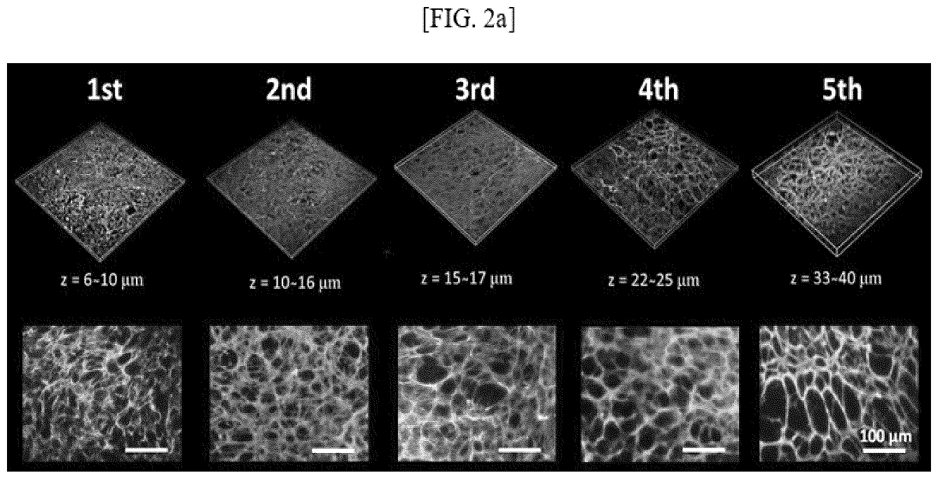

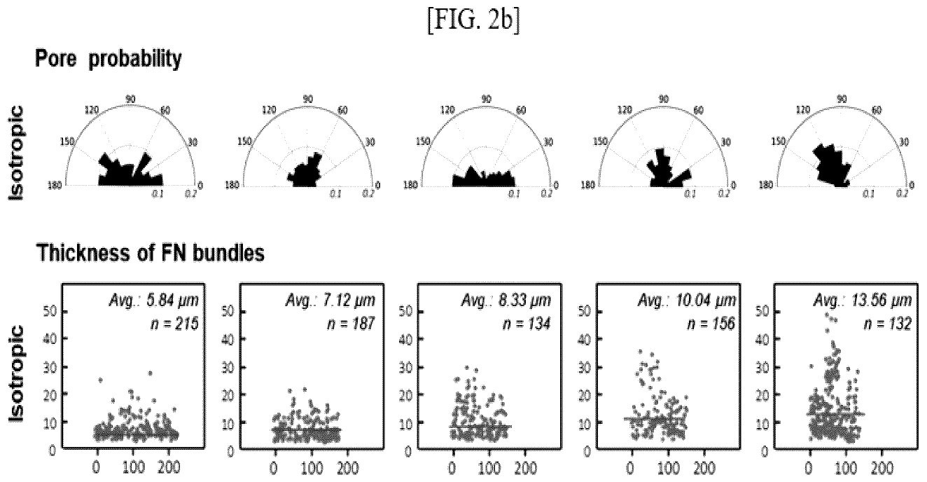

In order to confirm changes in the structure due to ECM layer stacking, the topology of samples in which the cell-culture and cellular component (CC) removal processes were performed once, twice, three times, four times, or five times was measured. The results are shown in FIGS. 2a and 2b.

Test results showed that the thickness (Z) of the stacked ECM was 6-10 μm when the cell-culture and CC removal processes were performed once, 10-16 μm when performed twice, 15-17 μm when performed three times, 22-25 μm when performed four times, and 33-40 μm when performed five times, indicating that the ECM thickness increased as the cell-culture and CC removal processes were repeated. The size of fibronectin (FN) bundles within the sample was on average 5.84 μm when the processes were performed once, on average 7.12 μm when performed twice, on average 8.33 μm when performed three times, on average 10.04 μm when performed four times, and on average 13.56 μm when performed five times, likewise showing an increase as the cell-culture and CC removal processes were repeated, in the same pattern as ECM thickness.

2. Confirmation of CC Removal within ECM

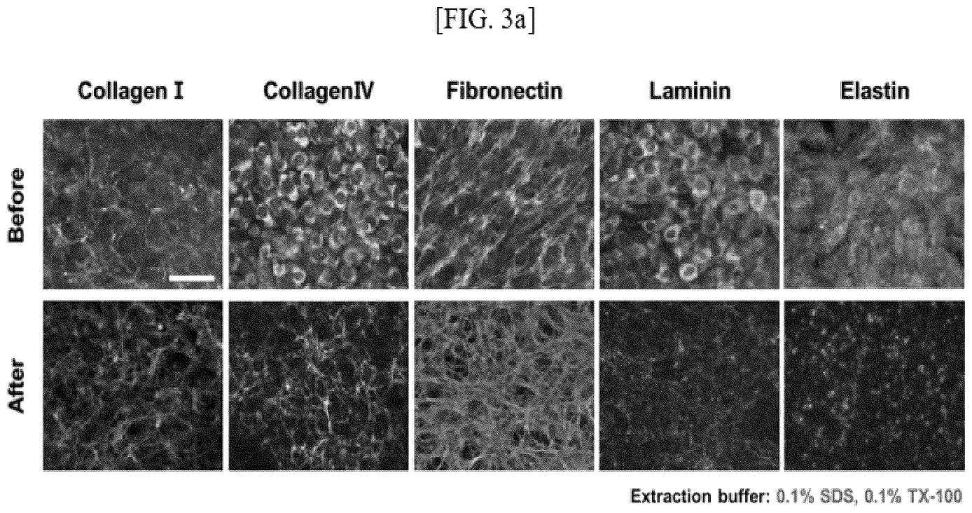

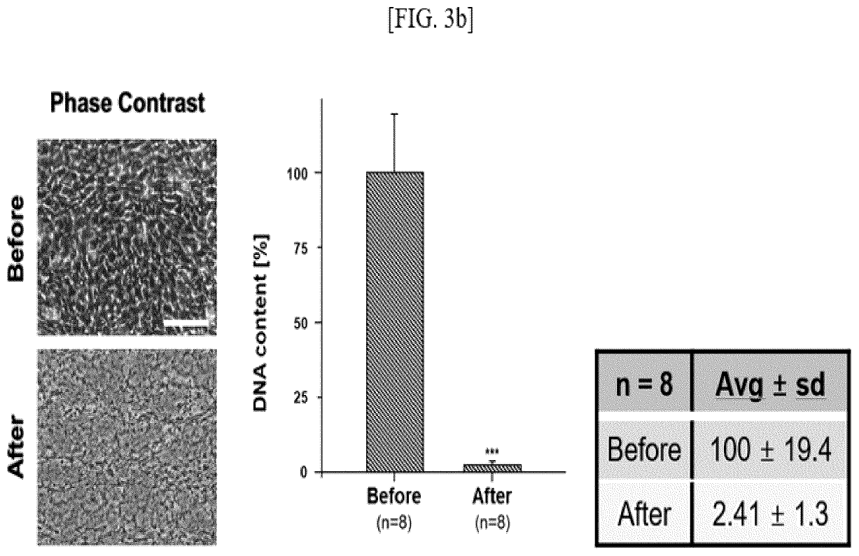

To confirm whether cellular components (CC) other than ECM were properly removed in the samples subjected to the cell-culture and CC removal processes, samples before and after the CC removal process were immunostained and compared. The results are shown in FIGS. 3a and 3b.

Test results showed that in the sample before the CC removal process, ECM components of collagen I, collagen IV, fibronectin, laminin, and elastin were stained together with cell nuclei, whereas in the sample after the CC removal process, only the cell nuclei were removed while all of the above ECM components remained as they were, confirming that only cellular components (CC) other than ECM were successfully removed through the CC removal process. Results of checking DNA concentration in the two samples also showed that cell nuclei and nucleic acids were well removed through the CC removal process.

### 3. Proteomic Analysis of Multilayer ECM

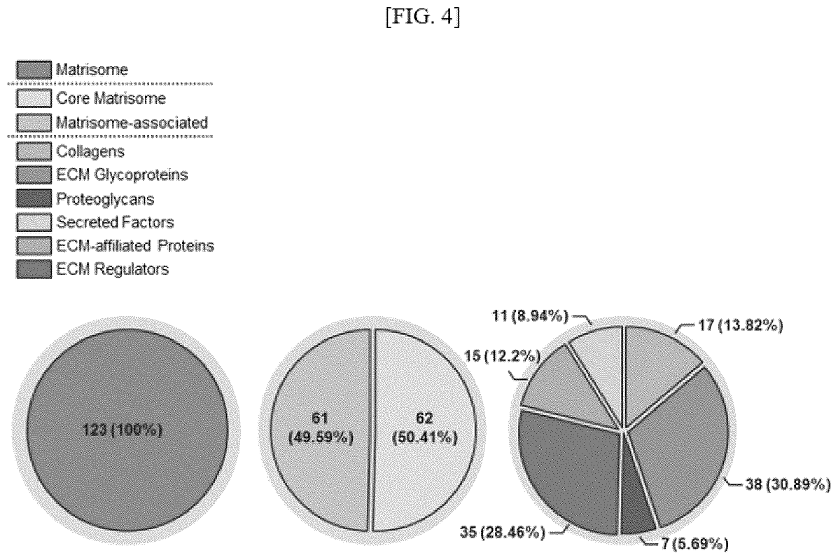

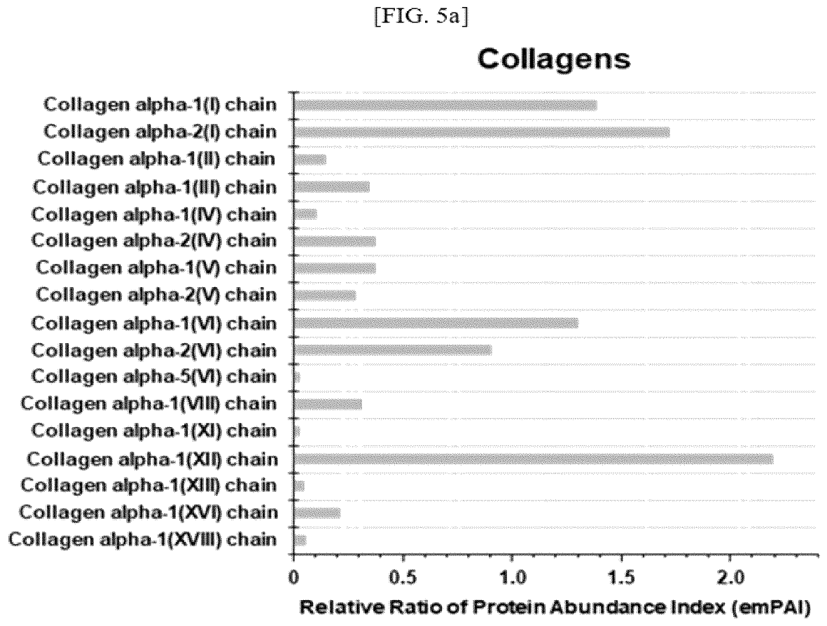

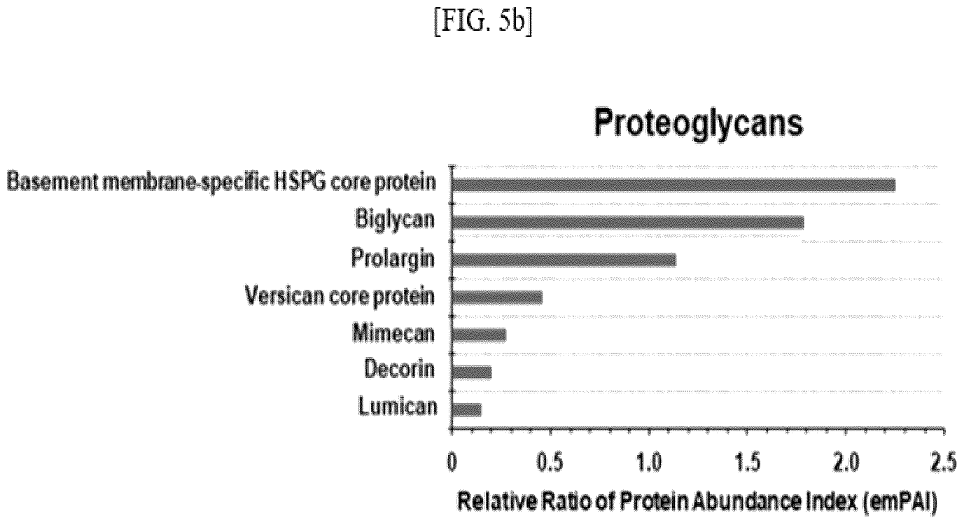

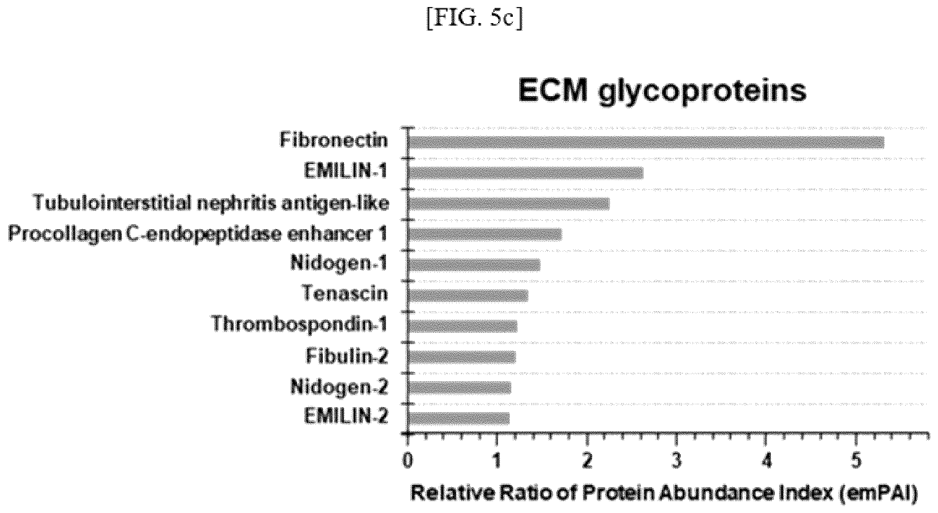

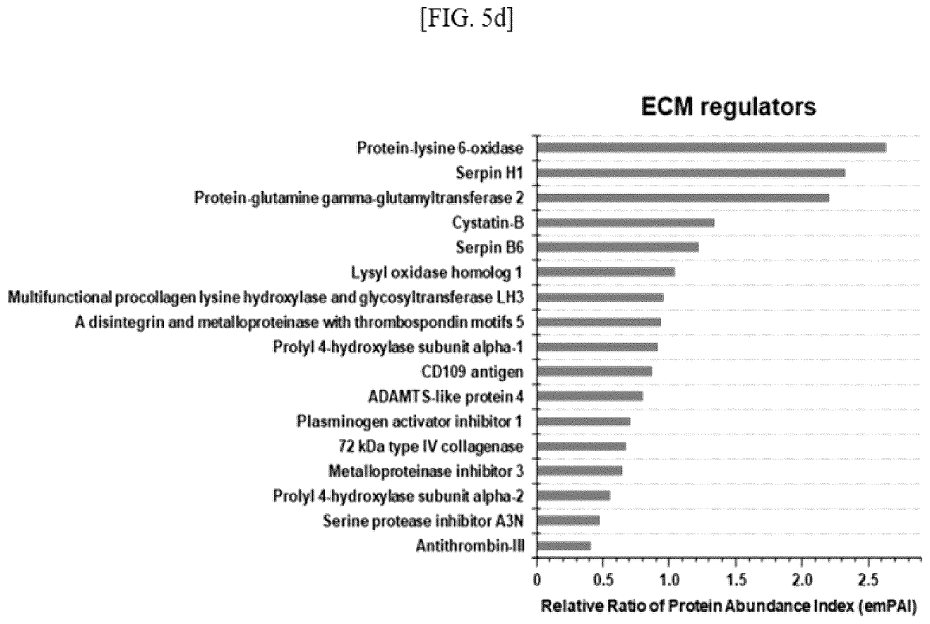

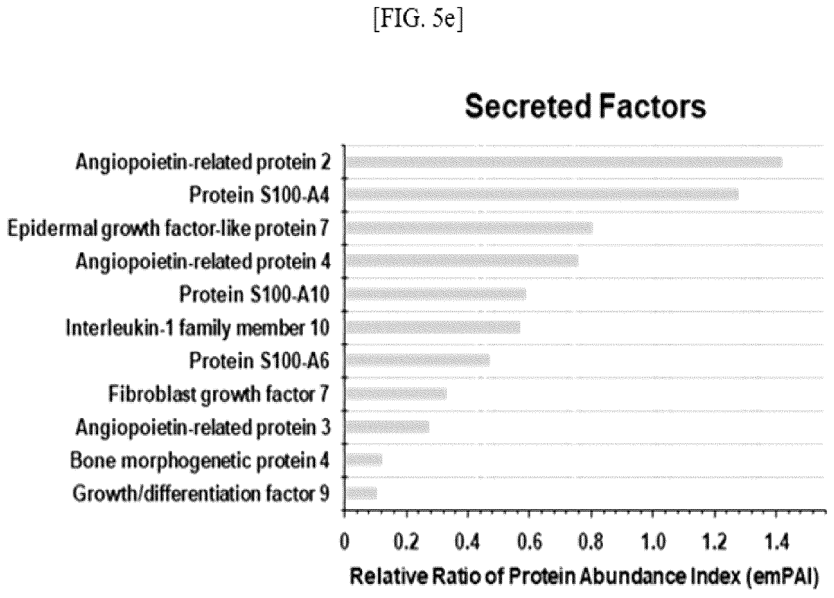

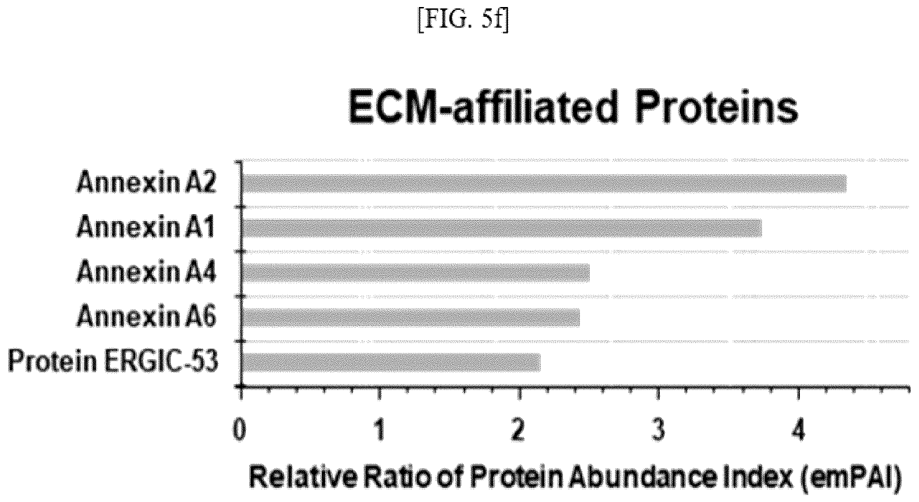

The results of proteomic analysis on the multilayer ECM sample for which the cell-culture and CC removal processes were repeated five times are shown in FIGS. 4 and 5.

FIG. 4 shows, by dividing, the matrisome encompassing ECM proteins and ECM-related proteins (ECM-associated factors) into core matrisome and matrisome-associated proteins, and shows how diverse matrisome components are contained in the cell-derived multilayer ECM of the present invention.

Test results showed that the multilayer ECM sample of the present invention included 123 proteins among the entire matrisome. Of these, 62 were core matrisome and 61 were matrisome-associated proteins. In detail, the core matrisome included 17 collagens, 38 ECM glycoproteins, and 7 proteoglycans, and the matrisome-associated proteins included 11 secreted factors, 15 ECM-affiliated proteins, and 35 ECM regulators.

FIGS. 5a to 5f show representative protein lists by each group, allowing relative quantitative comparison of the corresponding proteins. For this, the emPAI index formula used was referenced from prior literature in the field (Ishihama, Yasushi, et al. “Exponentially modified protein abundance index (emPAI) for estimation of absolute protein amount in proteomics by the number of sequenced peptides per protein” Molecular & Cellular Proteomics 4.9 (2005): 1265-1272.), and a publicly known DB was also used as the database for the corresponding matrisome analysis (Naba A, Clauser K R, Hoersch S, Liu H, Carr S A, and Hynes R O. The matrisome: in-silico definition and in-vivo characterization by proteomics of normal and tumor extracellular matrices. Molecular and Cellular Proteomics, 2012, 11(4):M111.014647. https://doi.org/10.1074/mcp.M111.014647). As a result of the analysis, in the core matrisome, collagens were found to be abundantly present mainly as the al chain of collagen XII, the α1 and α2 chains of collagen I, and the α1 and α2 chains of collagen VI; ECM glycoproteins were abundantly present mainly as fibronectin and EMILIN-1; and proteoglycans were abundantly present mainly as basement membrane-specific HSPG core protein, biglycan, prolargin, and versican. Among matrisome-associated proteins, secreted factors were abundantly present mainly as angiopoietin-related protein 2, S100A4, and EGF-like protein 7; ECM-affiliated proteins were abundantly present mainly as annexin A2 and A1; and ECM regulators were abundantly present mainly as protein lysine 6 oxidase, serpin H1, and transglutaminase 2. This means that the ECM structure of the present invention is constituted by including a very diverse set of ECM components, and that it can mimic natural ECM.

### 4. Confirmation of Structure and Physical Properties of Glycated Multilayer ECM

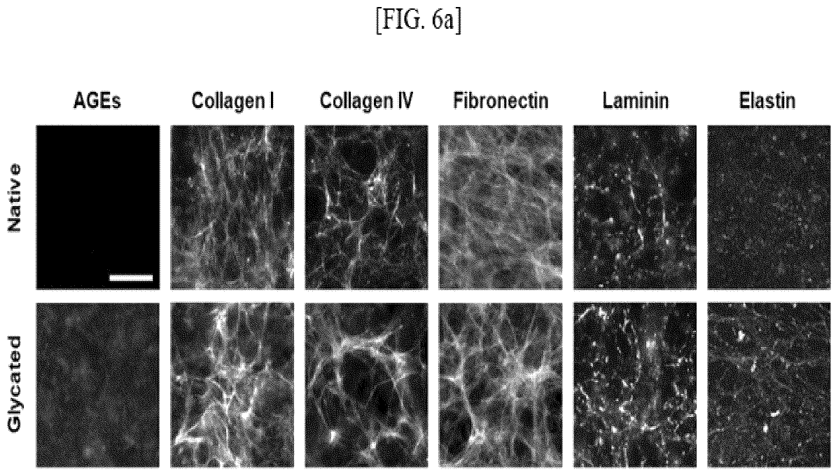

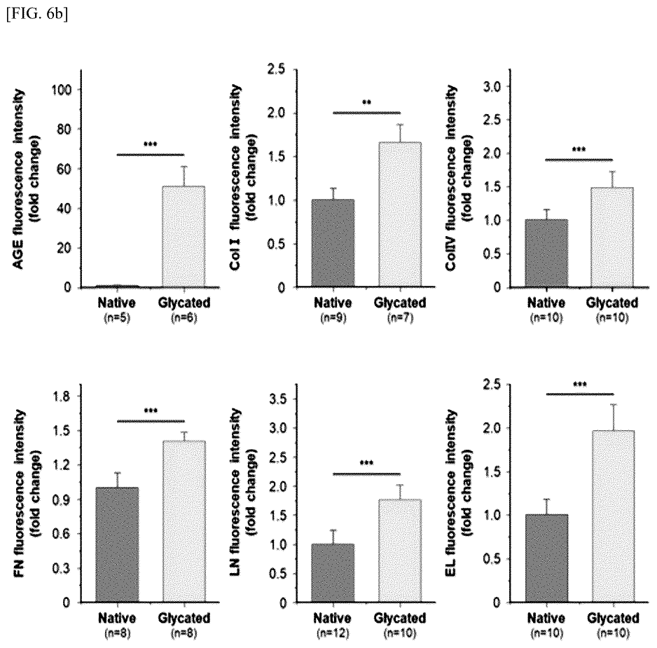

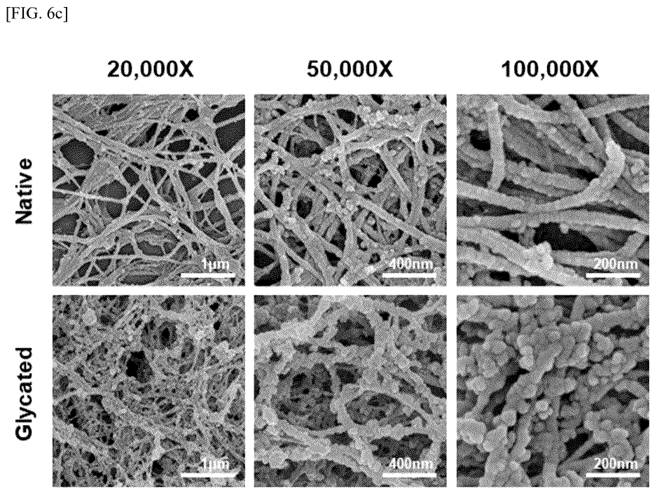

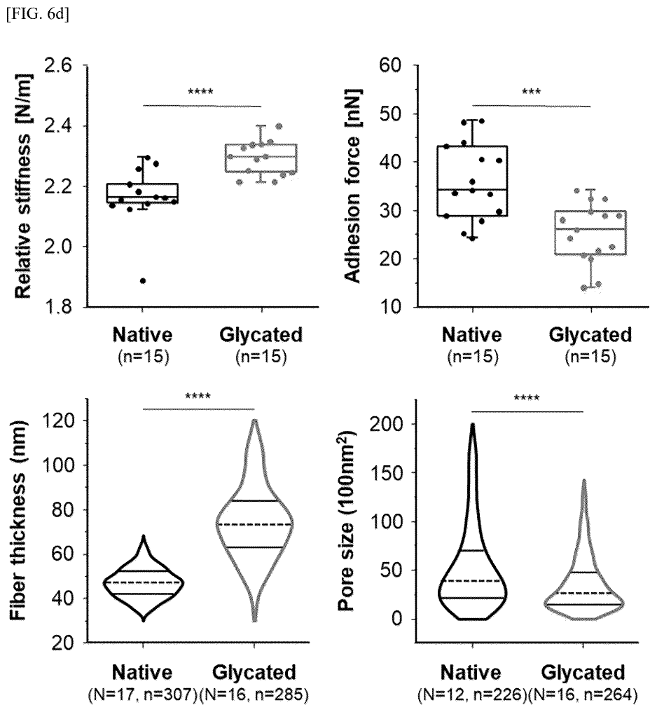

Changes before and after performing a glycation process with ribose on the multilayer ECM sample of the present invention were analyzed, and the results are shown in FIGS. 6a to 6d.

Test results by immunostaining confirmed that a large amount of advanced glycation end-products (AGEs) were formed and accumulated in the ECM, and that the fluorescence intensity of ECM components (collagen I, collagen IV, fibronectin, laminin, and elastin) significantly increased due to glycation. In addition, according to physical analysis results, stiffness and fiber thickness in the sample were significantly increased due to glycation, and adhesion force and pore size were significantly decreased. This means that the ECM structure was physically reinforced by glycation.

### 5. Proteomic Analysis of Glycated Multilayer ECM

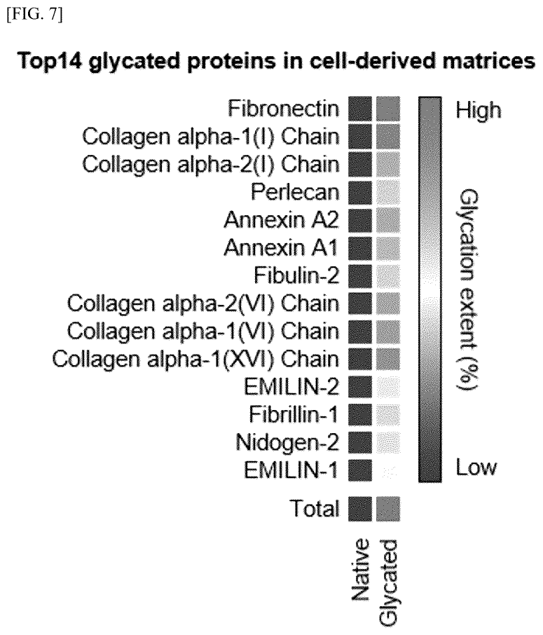

The results of proteomic analysis after glycation treatment with ribose, or glucose, of a multilayer ECM sample for which the cell-culture and CC removal processes were repeated five times are shown in FIGS. 7 to 15. The “degree to which glycation progressed” was compared/analyzed based on the total amount of all advanced glycation end-products confirmed by proteomic analysis for each ECM protein, and a sample not subjected to glycation treatment as a control was indicated as “native”.

FIGS. 7 to 11 are results from glycation treatment with ribose, wherein FIG. 7 shows, as a result of conducting proteomic analysis after glycation treatment of cell-derived ECM having a multilayer structure, the top 14 proteins according to the degree to which glycation progressed among matrisomes present in the sample. As a result of the analysis, even with the same glycation induction period (7 days), a relatively large amount of accumulation of advanced glycation end-products was confirmed in fibronectin, collagen I, and collagen XVI, indicating that even if the period during which the glycation process is induced is the same, the degree to which advanced glycation end-products are formed can differ. The amount of advanced glycation end-products according to the period for which glycation was treated increased overall.

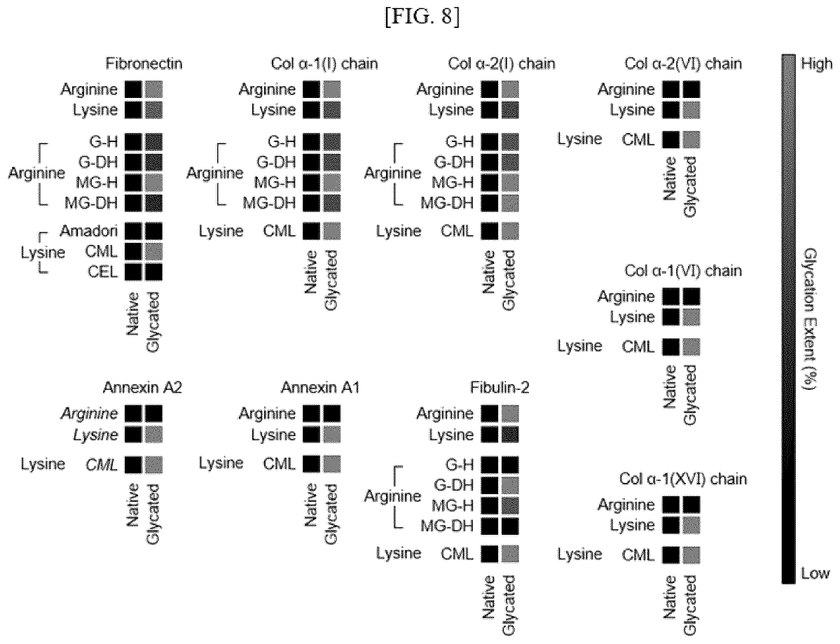

FIG. 8 shows advanced glycation end-products confirmed by proteomic mass analysis. For each protein constituting the cell-derived ECM used as the sample, amino acid residues at which advanced glycation end-products are present and the types of advanced glycation end-products at each amino acid residue were indicated together. The glycation process is known to induce the formation of advanced glycation end-products targeting only lysine and arginine residues of proteins, and as such, it can be confirmed that formation of advanced glycation end-products was not detected at amino acid residues other than these. In addition, it was found that the types of advanced glycation end-products formed differ depending on the amino acid residue. For example, at arginine residues, types such as G-H, G-DH, MG-H, and MG-DH are formed, and at lysine residues, types such as Amadori product, CML, and CEL are formed. Accordingly, it could be seen that even with the same glycation induction period, the pattern of formation of advanced glycation end-products can differ for each ECM protein.

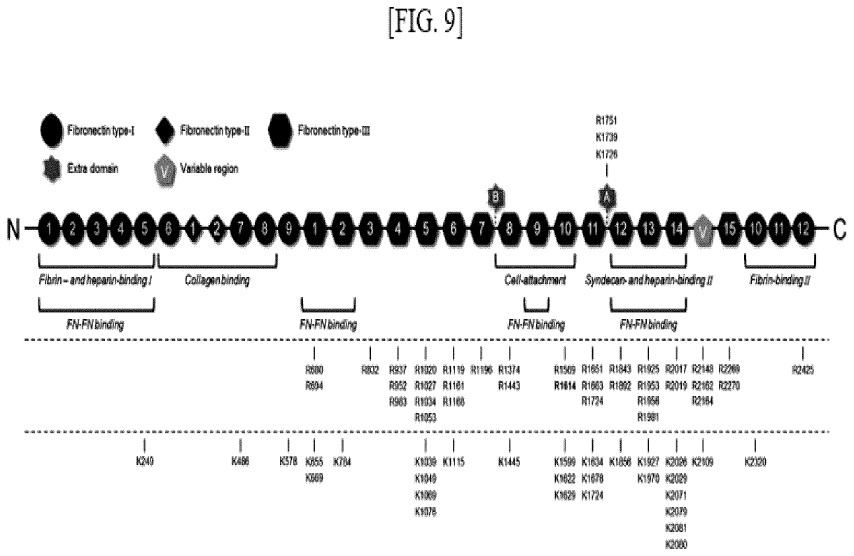

FIG. 9 shows amino acid residues at which advanced glycation end-products were confirmed in the full-length protein of fibronectin. In the case of fibronectin, it is composed of a total of three types (type-I, type-II, and type-III) of modules, extra domain, and variable region, etc., and each module gathers to form a functional domain. For example, modules 1 to 5 gather to form a fibrin- and heparin-binding domain, which means that when fibrin or heparin interacts with fibronectin, it can bind to modules 1 to 5 of fibronectin. Referring to FIG. 9, it is possible to check what functional domains are formed by the gathering of respective modules in fibronectin and in collagen type I. FIG. 9 also shows the positions of residues among all amino acid residues constituting fibronectin at which formation of advanced glycation end-products was confirmed by the ribose glycation process. Those beginning with R- are positions of arginine, and those beginning with K- are positions of lysine. For example, K249 means that among the amino acid residues constituting fibronectin, there is a lysine residue at the 249th position, and it can be confirmed that formation of an advanced glycation end-product occurred at this position.

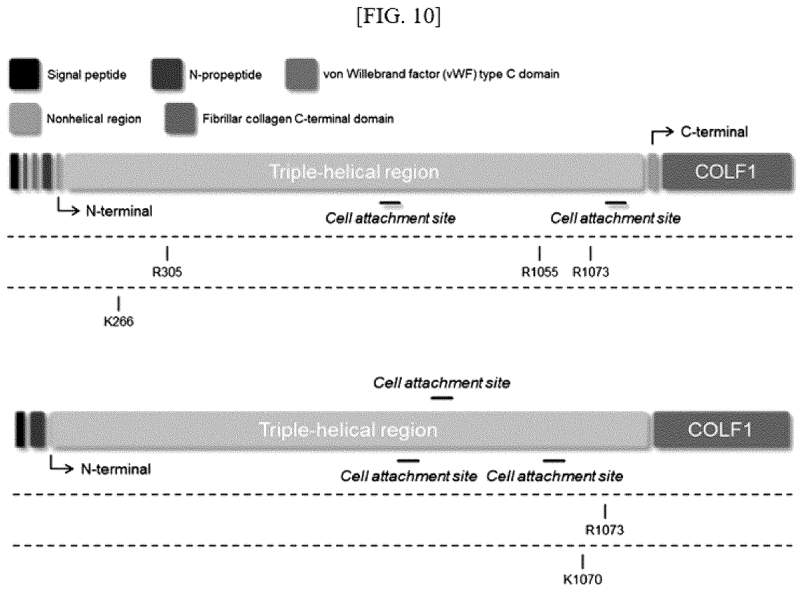

FIG. 10 shows amino acid residues at which advanced glycation end-products were confirmed in the full-length protein of collagen type I. The symbols and indication method in the drawing are the same as in FIG. 9.

Combining FIGS. 9 and 10, it can be confirmed that the positions of amino acid residues at which advanced glycation end-products are formed by the glycation process can differ depending on the ECM protein, and that, relative to collagen I, far more positions showed formation of advanced glycation end-products in fibronectin.

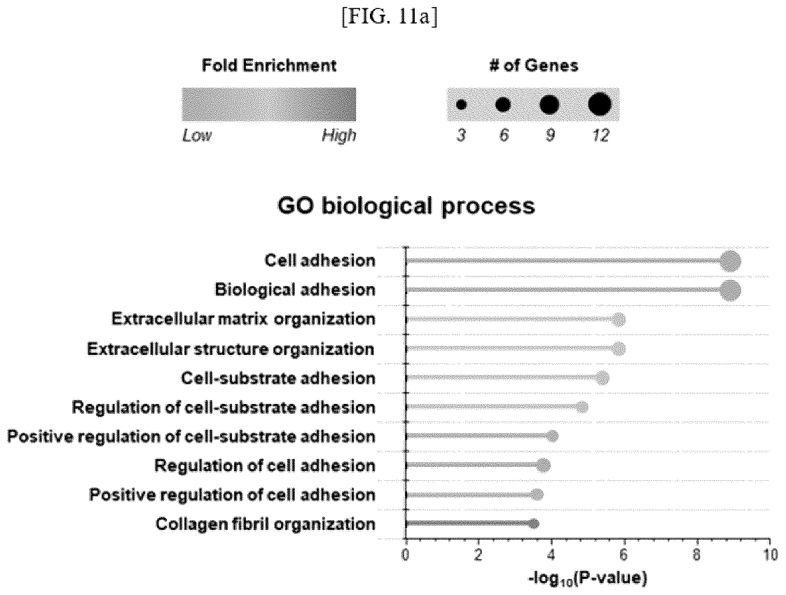

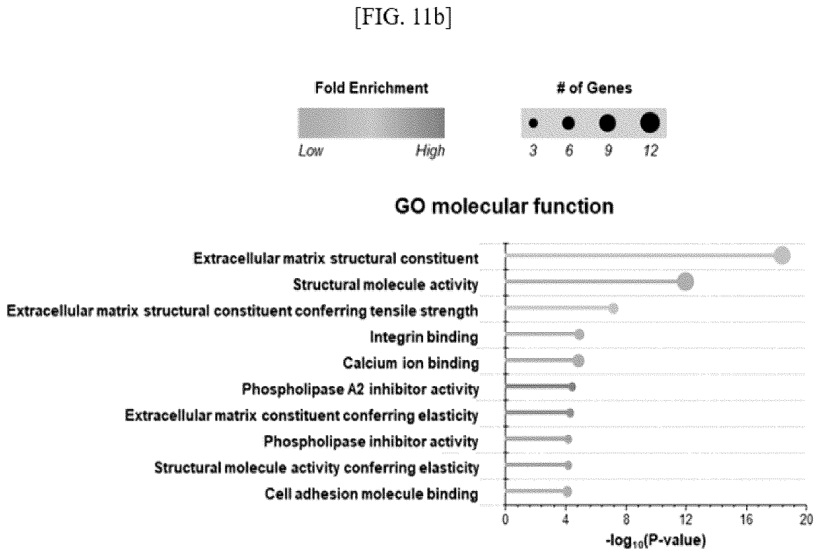

FIGS. 11a and 11b are GO (Gene ontology) analysis results using the top 14 glycated proteins described in FIG. 7. Referring to FIG. 11, it can be seen that ECM proteins modified by the glycation process are proteins that play an important role in cell adhesion. This means that these proteins are, at the molecular level, involved in the ECM structure and perform important functions in binding with cell adhesion molecules including integrin.

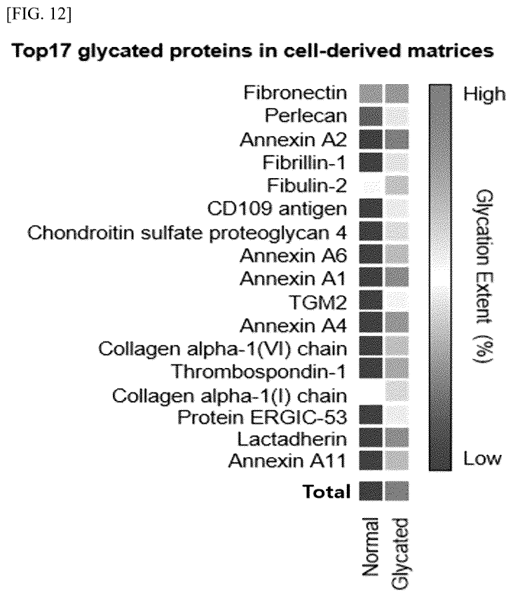

FIGS. 12 to 15 are results from glycation treatment with glucose, wherein FIG. 12 shows, as a result of conducting proteomic analysis after glycation treatment of cell-derived ECM having a multilayer structure, the top 17 proteins according to the degree to which glycation progressed among matrisomes present in the sample. This shows that even if the period for which glucose is treated is the same depending on the ECM protein, that is, even if the period during which the glycation process is induced is the same, the degree to which advanced glycation end-products are formed can differ. A relatively large amount of accumulation of advanced glycation end-products was confirmed in fibronectin, annexin A1, annexin A2, annexin A4, and lactadherin. The “total” in the last row is a result showing, by comparing the total amount of advanced glycation end-products present in all ECM proteins in which the glycation process was induced for each time, that the amount of advanced glycation end-products increases overall according to the period for which glucose is treated. When compared with FIG. 7, which is the result using ribose, it can be confirmed that the kinds of ECM proteins in which considerable changes were induced by glycation are very different. This means that the composition of proteins that become the main targets can vary depending on the type of sugar molecule that causes the glycation process, and that the speed of the glycation reaction and the degree to which changes are induced can also differ.

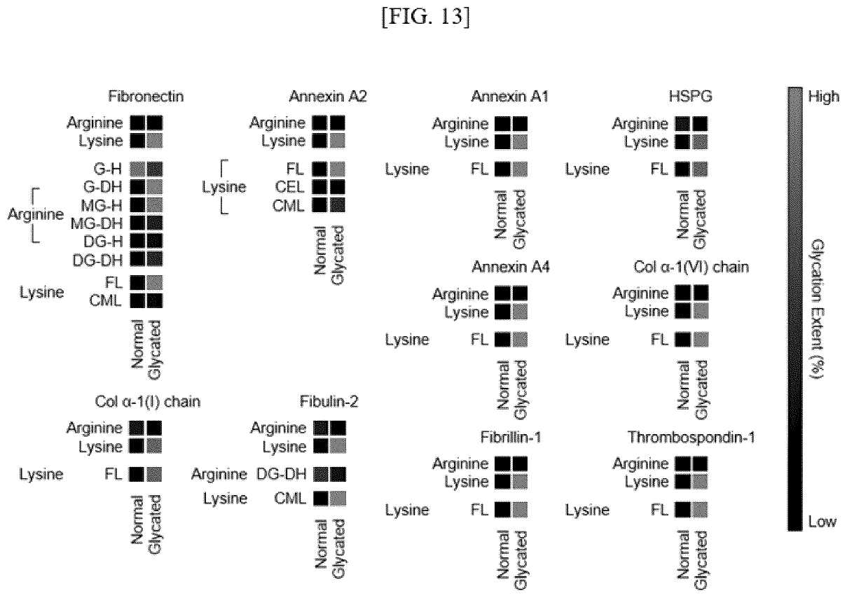

FIG. 13 is a result showing advanced glycation end-products confirmed by proteomic mass analysis. For each protein constituting the cell-derived ECM used as the sample, amino acid residues at which advanced glycation end-products are present and the types of advanced glycation end-products at each amino acid residue were indicated together. As with ribose glycation, it can also be confirmed that in the glycation process using glucose, formation of advanced glycation end-products was not detected at amino acid residues other than lysine and arginine residues of proteins. In addition, there exist various kinds of advanced glycation end-products, and the kinds of advanced glycation end-products formed differ depending on the amino acid residue according to the reaction, and the glycation process through glucose produces kinds of advanced glycation end-products different from the glycation process through ribose in FIG. 8. For reference, in glucose glycation, advanced glycation end-products such as G-H, G-DH, MG-H, MG-DH, IB, D-GH, DG-DH, and Argp are mainly formed at arginine residues, and advanced glycation end-products such as FL, CEL, Pyr, CML, AFGP, and GP are mainly formed at lysine residues. Furthermore, as with ribose, it can be confirmed that even if exposed to glucose for the same period and the glycation process is induced, the pattern by which advanced glycation end-products are formed can differ for each ECM protein. However, it shows that in most proteins, advanced glycation end-products are mainly accumulated at lysine rather than arginine, and furthermore, it could be seen that a more monotonic pattern of change was shown than with glycation due to ribose.

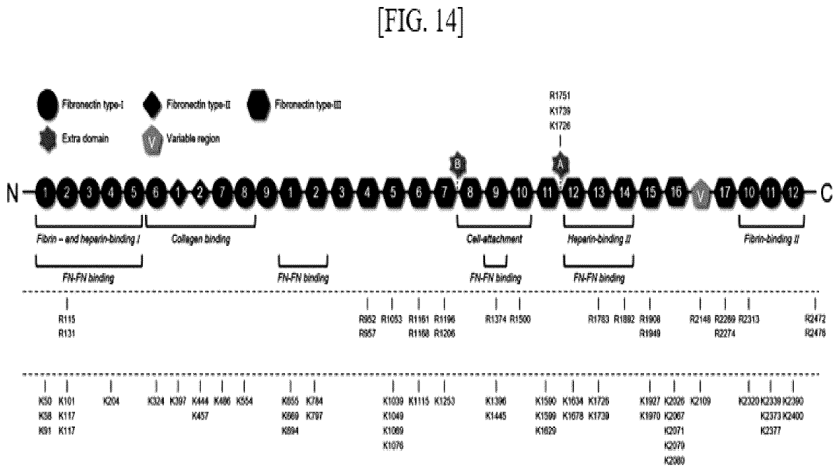

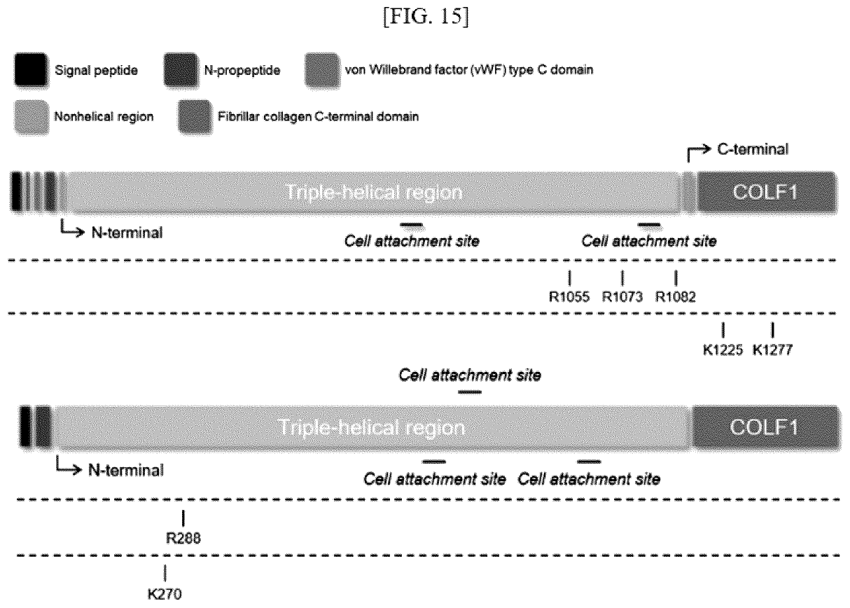

FIGS. 14 and 15 are results in which all amino acid residues at which advanced glycation end-products were confirmed in the full-length proteins of fibronectin (Fn) and collagen type I (Col I) due to the glucose glycation process were indicated. First, FIG. 14 is a drawing representing full-length fibronectin. In the case of fibronectin, it is composed of a total of three types (type-I, type-II, and type-III) of modules, extra domain, and variable region, etc., and each module gathers to form a functional domain. For example, modules 1 to 5 gather to form a fibrin- and heparin-binding domain, which means that when fibrin or heparin interacts with fibronectin, it can bind to modules 1 to 5 of fibronectin. Referring to FIG. 14, it is possible to check what functional domains are formed by the gathering of the respective modules in fibronectin and collagen type I. FIG. 14 also shows the positions of residues among all amino acid residues constituting fibronectin at which formation of advanced glycation end-products was confirmed by the glucose glycation process. Those beginning with R- are positions of arginine, and those beginning with K- are positions of lysine. For example, K204 means that among the amino acid residues constituting fibronectin, there is a lysine residue at the 204th position, and it shows that formation of an advanced glycation end-product was confirmed at this position. FIG. 15 is a drawing representing full-length collagen type I. The upper part shows the alpha 1 chain of collagen type I, and the lower part shows the alpha 2 chain of collagen type I. Likewise, in collagen type I as well, positions of amino acid residues at which functional domains and formation of advanced glycation end-products were confirmed were indicated.

Taken together, it can be confirmed that the positions of amino acid residues at which advanced glycation end-products are formed by the glycation process differ depending on the ECM protein. Relatively, far more positions showed formation of advanced glycation end-products in fibronectin than in collagen type I, and the glycation process due to ribose and the glycation process due to glucose formed advanced glycation end-products at different positions of amino acid residues even in the same ECM protein. That is, this indicates that even if the glycation process targets the same protein, the functional domains affected within the protein may differ depending on the glycation-inducing substance, and this means that even for the same glycation process, the direction and manner in which proteins are altered can differ depending on the sugar molecule.

### 6. Analysis of the Effect of Acrylate-Based Polymer Resin Coating in Multilayer ECM

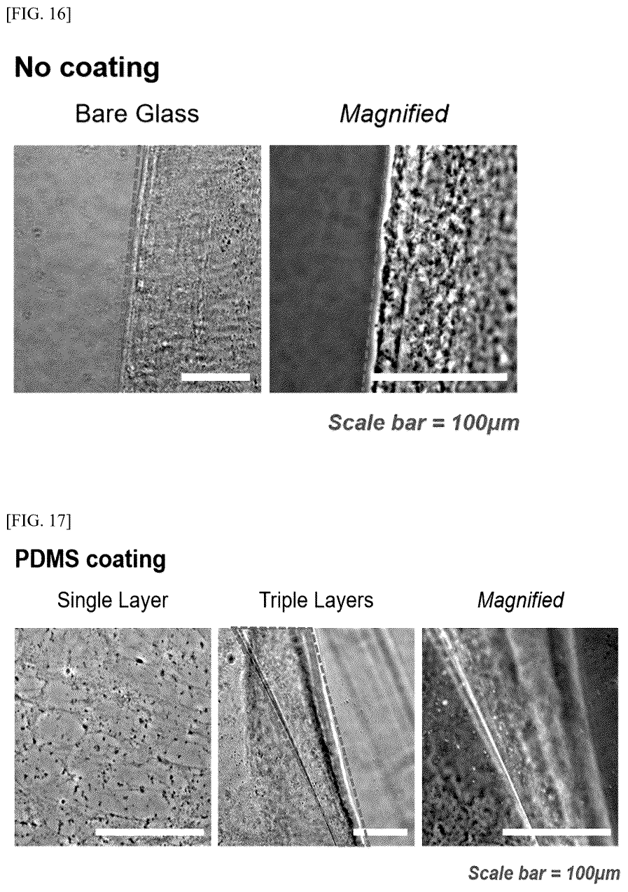

In the manufacture of multilayer ECM, coating with an acrylate-based polymer resin and coating with a non-acrylate-based polymer resin can cause different results. Experimental results for this are shown in FIGS. 16 and 17.

FIG. 16 shows a result showing the appearance of an extracellular matrix structure formed thereon when no polymer was coated after plasma treatment on a glass surface. It can be confirmed that even a single-layer extracellular matrix structure failed to adhere well on the glass surface and delaminated. The dotted line indicates the boundary line of the extracellular matrix structure attached to the glass surface, and based on this line, the extracellular matrix on the left side peeled off from the surface and overlapped with the structure on the right side.

FIG. 17 is a result of stacking an extracellular matrix structure thereon after coating the glass with PDMS (polydimethylsiloxane) as a non-acrylate polymer resin after plasma treatment. Unlike when nothing was coated on the glass, when PDMS was coated, a single-layer extracellular matrix structure adhered well on the glass surface, and the nanoscale fiber network within the extracellular matrix maintained its form well. However, in the subsequent process of stacking the extracellular matrix structure in multiple layers, the lowermost structure showed unstable adhesion, and in particular, when the third layer was stacked, a part was completely detached from the glass surface due to the contractile force pulled by the recultured cells. The region of the detached extracellular matrix structure was indicated by dotted lines. Compared with acrylate-based polymer resin coating, this difference is considered to be because, unlike non-acrylate-based polymers, acrylate-based polymers have highly polar ester groups and therefore have higher adhesion strength. In addition, acrylate-based polymers have a good balance between flexibility and rigidity, which is advantageous for supporting structures or maintaining structural deformation, and at the same time can provide appropriate long-term stability. As a result, when using acrylate-based polymers, it is expected that the weak adhesive force between the glass surface and the extracellular matrix structure can be compensated, thereby enabling stable manufacture of multilayer extracellular matrices.

Although the present disclosure has been described in detail with reference to the specific features, it will be apparent to those skilled in the art that this description is only of a preferred embodiment thereof, and does not limit the scope of the present disclosure. Thus, the substantial scope of the present disclosure will be defined by the appended claims and equivalents thereto.

### INDUSTRIAL APPLICABILITY

The in-vitro aging model including the multilayer extracellular matrix of the present invention faithfully implements the multilayer ECM structural form, and thus can mimic the three-dimensional structure of various tissues depending on the type of cells applied, and in particular, since it is similar to the ECM form of skin tissue, it is expected to be greatly utilized for skin aging research.

1. A method for manufacturing a multilayer extracellular matrix structure, comprising:
(a) a step of coating a cell adhesion molecule on a hydrophobic substrate;
(b) a step of culturing cells on the substrate of step (a);
(c) a step of removing components other than extracellular matrix from the substrate of step (b); and
(d) a step of glycating the extracellular matrix obtained in step (c).

2. The method of claim 1, wherein the hydrophobic substrate is a glass substrate.

3. The method of claim 1, wherein the cell adhesion molecule is one or more selected from the group consisting of collagen, fibronectin, laminin, elastin, cadherin, integrin, selectin, proteoglycan, and glycosaminoglycan.

4. The method of claim 1, wherein the cells in step (b) are stromal cells.

5. The method of claim 4, wherein the stromal cells are one or more selected from the group consisting of fibroblasts, chondroblasts, osteoblasts, neuroglial cells, adipocytes, macrophages, and plasma cells.

6. The method of claim 1, wherein steps (b) and (c) are repeated three or more times.

7. The method of claim 1, wherein the glycation is performed using ribose or glucose.

8. The method of claim 1, wherein the glycation is performed three or more days.

9. The method of claim 1, wherein the components other than the extracellular matrix in step (c) are excluded, including nucleic acids and lipids.

10. A multilayer extracellular matrix structure, manufactured by the method of claim 1, comprising multilayer extracellular matrix formed on a substrate.

11. The multilayer extracellular matrix structure of claim 10, wherein the substrate is a glass substrate.

12. The multilayer extracellular matrix structure of claim 10, wherein the multilayer comprises three or more layers.

13. The multilayer extracellular matrix structure of claim 10, wherein the extracellular matrix is stromal-cell-derived extracellular matrix.

14. The multilayer extracellular matrix structure of claim 13, wherein the stromal cells are one or more selected from the group consisting of fibroblasts, chondroblasts, osteoblasts, neuroglial cells, adipocytes, macrophages, and plasma cells.

15. The multilayer extracellular matrix structure of claim 10, wherein the structure does not comprise nucleic acids and lipids.

16. The multilayer extracellular matrix structure of claim 10, wherein the structure is for use in tissue aging research.

17. A screening method for a candidate substance for inhibiting tissue aging, comprising:
(a) a step of manufacturing the multilayer extracellular matrix structure of claim 10 including multilayer extracellular matrix formed on a substrate;
(b) a step of treating the structure with a candidate substance for inhibiting tissue aging; and
(c) a step of confirming changes in the structure.

18. The screening method of claim 17, wherein the tissue is skin tissue.
